(19) United States
(12) Patent Application Publication
(10) Pub. No.: US 2020/0283389 A1
(43) Pub. Date: Sep. 10, 2020

(54) IMPROVED PROCESS FOR MAKING CROTONYLAMINOPYRIDINIES

(71) Applicant: Intervet Inc.

(72) Inventors: Michael Berger, Wiesbaden (DE); Hans Peter Niedermann, Bubenheim (DE); Tobias Kappesser, Heidesheim (DE); Stephan Veit, Budenheim (DE); Heiko Bothe, Saulheim (DE); Marcus Knell, Ockenheim (DE); Christophe Pierre Alain Chassaing, Ingelheim am Rhein (DE)

(73) Assignee: Intervet Inc., Madison, NJ (US)

(21) Appl. No.: 16/063,944

(22) PCT Filed: Dec. 22, 2016
(86) PCT No.: PCT/EP2016/082295

§ 371 Date: Jun. 19, 2018

(30) Foreign Application Priority Data
Dec. 23, 2015 (EP) 15202565.6
Jun. 8, 2016 (EP) 16173553.5

(51) Int. Cl. C07D 213/75; C07D 213/74

(52) U.S. Cl. CPC C07D 213/75; C07D 213/74

(57) ABSTRACT

An improved process for producing a compound of Formula (I).

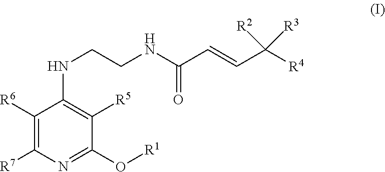

### BACKGROUND

International Publication No. WO 2012/041873 discloses certain N-heteroaryl compounds and processes to make the same. For example, the following transformations are disclosed:

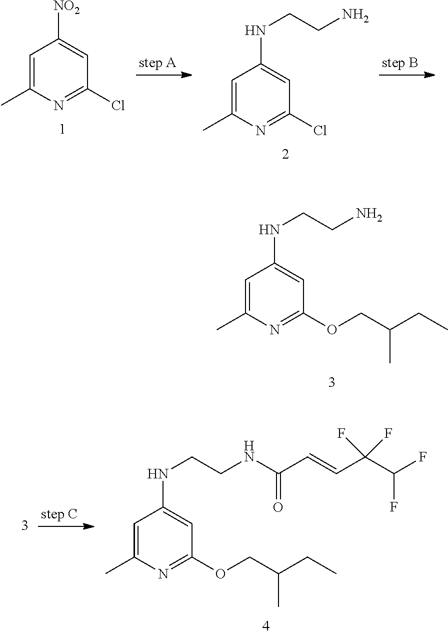 [SMILES: Cc1cc([N+](=O)[O-])cc(Cl)n1 ; Cc1cc(NCCN)cc(Cl)n1 ; CCC(C)COc1cc(NCCN)cc(C)n1 ; CCC(C)COc1cc(NCCNC(=O)/C=C/C(F)(F)C(F)F)cc(C)n1]

To complete step A, five extractions were required to isolate compound 2. In step B, a DMSO/diphenylether solvent system was used which required removal by chromatography. Moreover, diphenylether is solid at room temperature, thus difficult to handle on a large scale. For step C, the purification of compound 4 was performed by preparative HPLC, which is not suitable for large scale production process. Moreover, this step utilizes oxalyl chloride to create an acid chloride and DCM and DMF as solvents. These materials are also unsuitable for a large scale production process for various environmental and health/safety concern. The yield described by the overall process was in the range of 11 to 47%.

WO2015/177179 discloses a process to make substituted crotonic acid compounds which are useful intermediates in the preparation of crotonylaminopyridines.

### SUMMARY OF THE INVENTION

An embodiment of the invention is a process for producing a compound of Formula (I)

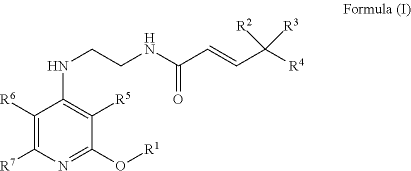

wherein:
R¹ is C₁-C₆ alkyl, wherein the C₁-C₆ alkyl is optionally substituted with halo;
R²-R⁴ are independently H, halo, C₁-C₆ alkyl or C₁-C₆ alkoxy, wherein the C₁-C₆ alkyl and the C₁-C₆ alkoxy are optionally substituted with halo, C₁-C₆ alkyl or C₁-C₆ alkoxy; and
R⁵-R⁷ are independently H or C₁-C₆ alkyl, wherein the C₁-C₆ alkyl is optionally substituted with halo;
comprising
a) reacting a compound of Formula (II)

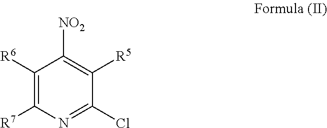

with ethylenediamine to form a compound of Formula (III)

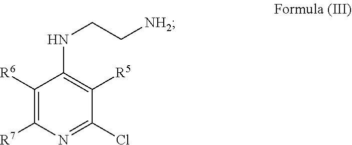

b) reacting in situ the compound of Formula (III) with R¹OX, wherein X is Na or K to form a compound of Formula (IV)

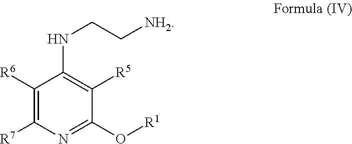

In another embodiment, the process, further comprises
step c) acylating the compound of Formula (IV) to give the compound of Formula (I).

An additional embodiment is a process for producing a compound of Formula (I)

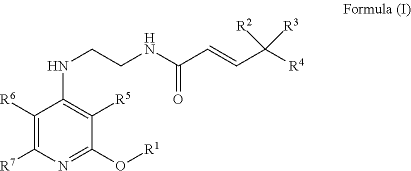

comprising
a) reacting a compound of Formula (V)

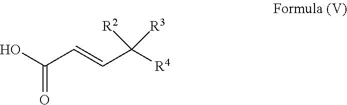

with a compound of Formula (IV) or a salt thereof

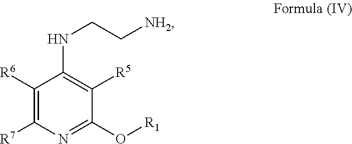

and an activating agent to give the compound of Formula (I).

### DETAILED DESCRIPTION

The subject application describes improved processes for making crotonylaminopyridine compounds.

In one embodiment, the process has been improved by eliminating the need to isolate and purify the N′-(2-chloro 4-pyridyl) ethane-1,2-diamine intermediate (Compound 2 in the Scheme 1 below).

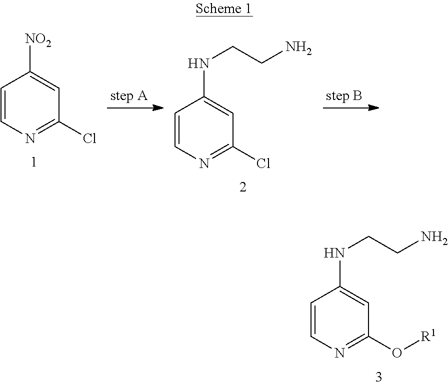

This change simplified the process by eliminating the need for repeated purification steps. Moreover, the change unexpectedly gave improved yields.

In another embodiment, the solvent for step A of Scheme 1 is an alcohol. In another embodiment, the solvent for step A is ethanol (EtOH). In another embodiment, the solvent for steps A and B of Scheme 1 is anisole.

In another embodiment, the process is improved by using an activating agent to conduct the acylation reaction (see step C in Scheme 2 below).

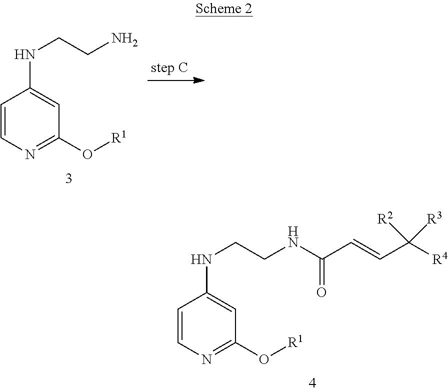

In another embodiment of the acylation reaction of step C, Scheme 2, the solvent is 2-methyl-THF and the activating agent is pivaloyl chloride. In another embodiment of the acylation reaction of step C, Scheme 2, the solvent is ethyl acetate (EtOAc) and the activating agent is propylphosphonic anhydride (T3P).

In another embodiment, the acylation reaction above is conducted with the salt of the N′-(2-alkoxy 4-pyridyl) ethane-1,2-diamine intermediate (see Scheme 3 below).

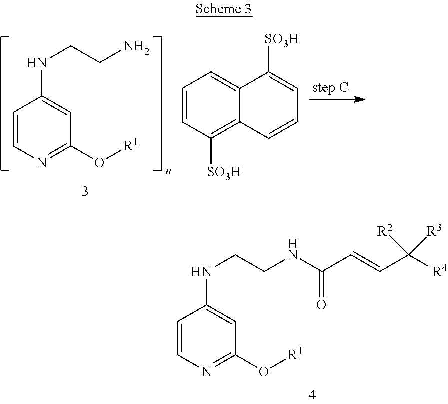

These improvements allowed for the isolation and purification of the final product by extraction or by extraction and crystallization instead of the chromatography used by the prior art process. They also led to improved yields of the overall reaction.

In another embodiment, the salt of the N′-(2-alkoxy 4-pyridyl) ethane-1,2-diamine intermediate of Scheme 3 is the hydrochloride (mono- and di-), sulfate, oxalate, di-mesylate, mono-tosylate or napadisylate salt.

An embodiment of the invention is a process for producing a compound of Formula (I)

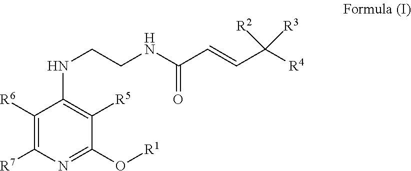

wherein:
R¹ is C₁-C₆ alkyl, wherein the C₁-C₆ alkyl is optionally substituted with halo;
R²-R⁴ are independently H, halo, C₁-C₆ alkyl or C₁-C₆ alkoxy, wherein the C₁-C₆ alkyl and the C₁-C₆ alkoxy are optionally substituted with halo, C₁-C₆ alkyl or C₁-C₆ alkoxy; and
R⁵-R⁷ are independently H or C₁-C₆ alkyl, wherein the C₁-C₆ alkyl is optionally substituted with halo;
comprising
a) reacting a compound of Formula (II)

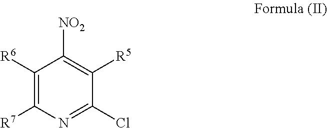

with ethylenediamine to form a compound of Formula (III)

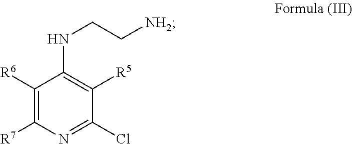

b) reacting in situ the compound of Formula (III) with R¹OX, wherein X is Na or K to form a compound of Formula (IV)

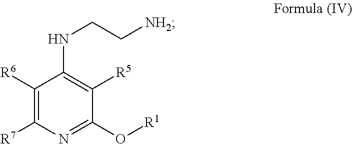

and
c) acylating the compound of Formula (IV) to give the compound of Formula (I).

Another embodiment of the invention is a process wherein R⁵, R⁶ and R⁷ are H.

Another embodiment of the invention is a process wherein R¹ is selected from the group consisting of methyl, ethyl, n-propyl and isopropyl.

Another embodiment of the invention is a process wherein R⁴ is F.

Another embodiment of the invention is a process wherein R² and R³ are independently selected from the group consisting of H, halo, CH₃, OCH₃ and CF₂OCH₃.

Another embodiment of the invention is a process wherein R⁵, R⁶ and R⁷ are H, R⁴ is F and R¹, R² and R³ are as defined below:

| R¹ | R² | R³ |
|---|---|---|
| CH₃ | F | F |
| CH₂CH₃ | F | F |
| CH₃ | OCH₃ | CF₃ |
| CH₃ | Br | F |
| CH₃ | F | H |
| CH₃ | H | CF₂OCH₃ |
| CH₃ | Cl | Cl |
| CH₃ | F | CH₃ |

Another embodiment of the invention is a process for producing a compound of Formula (I)

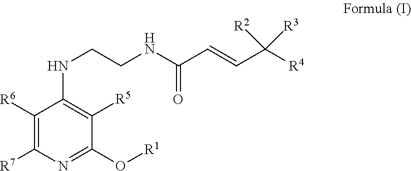

wherein:
R¹ is C₁-C₆ alkyl, wherein the C₁-C₆ alkyl is optionally substituted with halo;
R²-R⁴ are independently H, halo, C₁-C₆ alkyl or C₁-C₆ alkoxy, wherein the C₁-C₆ alkyl and the C₁-C₆ alkoxy are optionally substituted with halo, C₁-C₆ alkyl or C₁-C₆ alkoxy; and
R⁵-R⁷ are independently H or C₁-C₆ alkyl, wherein the C₁-C₆ alkyl is optionally substituted with halo;
comprising
a) reacting a compound of Formula (V)

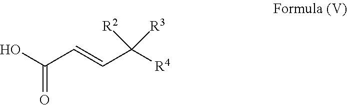

with a compound of Formula (IV) or a salt thereof

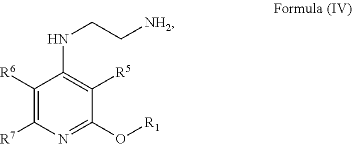

and an activating agent to give the compound of Formula (I).

In an embodiment of the invention, the compound of Formula (V) is initially reacted with the activating agent to give a mixed anhydride which is then reacted with the compound of Formula (IV).

In an embodiment of the invention, the activating agent is pivaloyl chloride or propylphosphonic anhydride and the mixed anhydride is a compound of formula (VI) or formula (VII), respectively.

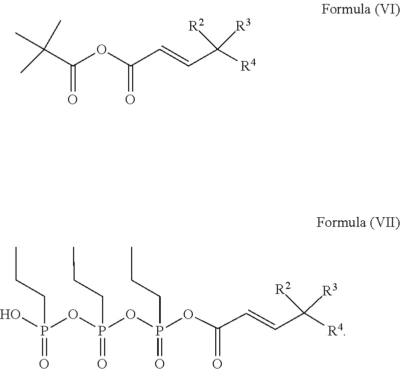

In an embodiment of the invention, the compound of Formula (V) and the compound of Formula (IV) are combined before the addition of the activating agent.

Another embodiment of the invention is a process wherein R⁵, R⁶ and R⁷ are H.

Another embodiment of the invention is a process wherein R¹ is selected from the group consisting of methyl, ethyl, n-propyl and isopropyl.

Another embodiment of the invention is a process wherein R⁴ is F.

Another embodiment of the invention is a process wherein R² and R³ are independently selected from the group consisting of H, halo, CH₃, OCH₃ and CF₂OCH₃.

Another embodiment of the invention is a process wherein R⁵, R⁶ and R⁷ are H, R⁴ is F and R¹ is ethyl, and R² and R³ are F.

Another embodiment of the invention is a process wherein R⁵, R⁶ and R⁷ are H, R⁴ is F and R¹ is methyl, and R² is methyl and R³ is F.

Another embodiment of the invention is a process wherein the salt of the compound of Formula IV is the hydrochloride, sulfate, oxalate, mesylate, tosylate or napadisylate salt.

Another embodiment of the invention is a process wherein the hydrochloride salt is the mono-hydrochloride salt or the di-hydrochloride salt.

Another embodiment of the invention is a process wherein the mesylate salt is the di-mesylate salt.

Another embodiment of the invention is a process wherein the tosylate salt is the mono-tosylate.

Suitable temperatures for step A in Scheme 1 range from room temperature to 70° C. In an embodiment, the temperature range is from 20° C. to 25° C. In another embodiment, the temperature range is from 50 to 60° C.

Ethylenediamine in step A is used preferably in excess. In an embodiment, the excess is 5 to 10-fold relative to 2-chloro-4-nitropyridine. In another embodiment, the excess is 6-fold relative to 2-chloro-4-nitropyridine.

In step A in Scheme 1 additional base can be added. In an embodiment, the added base is a carbonate salt of an alkali metal, preferably potassium carbonate. In an embodiment, the equivalent of added base ranges from 0.5 to 1 relative to 2-chloro-4-nitropyridine. In another embodiment the equivalent of added base ranges from 0.6 to 0.7 relative to 2-chloro-4-nitropyridine.

Suitable temperatures for step B in Scheme 1 range from 110 to 150° C., preferably from 120 to 125° C.

The alkoxide R¹OX can be added as a solid, a solution or as a slurry. In an embodiment, KOMe is added as solid, in another embodiment, KOMe is added as a slurry in anisole.

After completion of step B it is advantageous to perform a quench before work-up. In an embodiment, the reaction is quenched by the addition of water. In another embodiment, the reaction is quenched by the addition of sodium bicarbonate.

In an embodiment of the process of the invention, the product of step B is isolated as a salt by addition of an organic or inorganic acid. In an embodiment, the amount of acid added ranges from 1 to 2 equivalents relative to the product of step B. In another embodiment, 1 equivalent of acid is added. Preferably, the excess of ethylenediamine present is removed by distillation before crystallization of the salt. Preferably also water present is removed before crystallization of the salt. In an embodiment, excess ethylenediamine is removed by distillation under reduced pressure. In an embodiment, water is removed by distillation under reduced pressure.

In an embodiment, an alcohol is added before isolation of the product of step B as a salt. In another embodiment, the alcohol added is isopropanol.

Step C is done preferably in the presence of added base. The base is preferably an amine base. In an embodiment the base added is triethylamine. The suitable amount of base added ranges from 1 to 5 equivalents. In an embodiment 3.5 equivalents of base are added.

In an embodiment, a suitable temperature range for step C is from −5° C. to 25° C., preferably from −5° C. to 5°.

In the acylation reaction of step C a suitable molar ratio of the acid reactant of formula (V) and the amino reactant of formula (IV) or the salt thereof ranges from 0.9 to 1.1. In an embodiment, the molar ratio of (IV) and (V) is 1. The activating agent in the acylation reaction of step C can be used in a molar ratio of 0.9 to 1.2 relative to (IV). In an embodiment, the molar ratio of the activating agent relative to (IV) is 1.1.

After completion of the acylation of step C, the reaction is preferably quenched by the addition of water or diluted acid. In an embodiment, the reaction is quenched by the addition of sulfuric acid.

In an embodiment, the pH of the reaction mixture after quench is adjusted to 3-4 and the mixture is washed with an organic solvent, preferably ethyl acetate or 2-methyl THF. The pH of the aqueous phase is then adjusted to 6-8, preferably to 7-8 and the final product is isolated by extraction or crystallization.

The following definitions are provided to more clearly describe the invention.

“Alkyl” means an aliphatic hydrocarbon group which may be straight or branched and comprising about 1 to about 20 carbon atoms in the chain. In one embodiment alkyl groups contain about 1 to about 12 carbon atoms in the chain. In another embodiment alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. Non-limiting examples of suitable alkyl groups include methyl, ethyl, n-propyl, isopropyl, n-butyl, t-butyl, n-pentyl, heptyl, nonyl, or decyl.

“Halo” (or “halogeno” or “halogen”) means fluoro, chloro, bromo, or iodo groups. Preferred are fluoro, chloro or bromo, and more preferred are fluoro and chloro.

“Haloalkyl” means an alkyl as defined above wherein one or more hydrogen atoms on the alkyl are replaced by a halo group as defined above.

“Alkoxy” means an —O-alkyl group in which the alkyl group is as previously described. Non-limiting examples of suitable alkoxy groups include methoxy, ethoxy, n-propoxy, isopropoxy, n-butoxy and heptoxy. The bond to the parent moiety is through the ether oxygen.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

When used herein, the term “independently”, in reference to the substitution of a parent moiety with one or more substituents, means that the parent moiety may be substituted with any of the listed substituents, either individually or in combination, and any number of chemically possible substituents may be used.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

“Solvate” means a physical association of a compound of this invention with one or more solvent molecules. Non-limiting examples of suitable solvates include ethanolates, methanolates, and the like. “Hydrate” is a solvate wherein the solvent molecule is H₂O.

The term “salt(s)”, as employed herein, denotes acidic salts formed with inorganic and/or organic acids, as well as basic salts formed with inorganic and/or organic bases.

TEA is triethylamine.

DCM is dichloromethane.

DMF is dimethyl formamide.

DMSO is dimethyl sulfoxide.

THF is tetrahydrofuran.

EtOAc is ethyl acetate.

EtOH is ethanol.

T3P is propylphosphonic anhydride.

Tosylate salt refers to the product of the reaction between the free base diamine and p-toluenesulfonic acid.

Napadisylate salt refers to the product of the reaction between the free base diamine and naphthalene-1,5-disulfonic acid (also known as Armstrong's Acid).

Activating agent—Non-limiting examples of activating agents are pivaloyl chloride and propylphosphonic anhydride and the like.

When a compound of Formula (V)

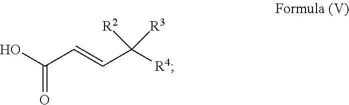

is reacted with the activating agents pivaloyl chloride or propylphosphonic anhydride, the mixed anhydride compounds of formula (VI) or formula (VII) respectively are formed:

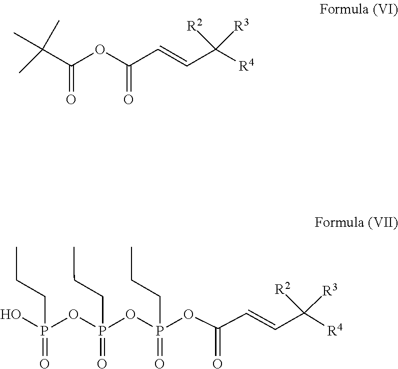

In situ means “locally”, “on site”. In the context of a chemical process, it means that the subsequent reaction is carried out without separation or purification of the products of the previous reaction.

### EXAMPLES

### Example 1: Synthesis of N′-(2-methoxy-4-pyridyl)ethane-1,2-diamine

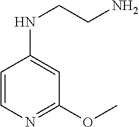

Comparative Example using stepwise reaction with isolation and purification of step a) product by pH-driven extraction.

### Step a): N′-(2-chloro-4-pyridyl)ethane-1,2-diamine

2-Chloro-4-nitropyridine (100 g, 0.61 mol) was dissolved in ethanol (1000 ml). Ethylenediamine (297 g, 4.9 mol) was added over a period of 2 hours under cooling keeping the temperature below 20° C. The mixture was stirred overnight at ambient temperature. The mixture was concentrated under reduced pressure, water (500 ml) was added to the residue and the pH of the mixture was adjusted to 13 by the addition of NaOH (6M). The resulting mixture was extracted with 2-methyl-THF (3×250 ml).

The combined organic extracts were extracted with HCl (1M, 2×300 ml), where in each extraction step the pH was adjusted to 7 (by addition of 6M HCl or 6M NaOH). The aqueous extracts were combined, the pH was adjusted to 13 (by addition of NaOH 6M). The resulting mixture was extracted with 2-methyl-THF (3×250 ml). The combined organic extracts were washed with brine (200 ml) dried (MgSO₄) and evaporated to dryness to yield 38.7 g of a solid.

¹H-NMR (300 MHz, DMSO-d₆): 7.77 (m, 1H), 6.88 (m, 1H), 6.48 (m, 2H), 3.03 (q, J=6.1 Hz, 2H), 2.66 (t, J=2.7 Hz, 2H); MS 172.1 (M+1).

### Step b): N′-(2-methoxy-4-pyridyl)ethane-1,2-diamine

Anisole (12 ml) was heated to 50° C., then the product of step a) (2.2 g, 12.8 mmol) was added, followed by sodium methoxide (2.98 g, 38.5 mmol) and the mixture was heated to reflux and stirred under reflux overnight. Water (1 ml) was added to the mixture, which was concentrated under reduced pressure. Water (20 ml) was added to the residue, the mixture was saturated with NaCl and extracted with 2-methyl-THF (3×20 ml). The combined organic layers were washed with brine (2×), dried (MgSO₄), and the solvent was removed under reduced pressure followed by freeze drying to yield 1.61 g of an oil which contains N′-(2-methoxy-4-pyridyl)ethane-1,2-diamine and N′-(2-chloro-4-pyridyl)ethane-1,2-diamine in a ratio of 3 to 10.

The NMR analysis (300 MHz, DMSO-d₆) of this oil showed a set of two signals:

Set a)—N′-(2-chloro-4-pyridyl)ethane-1,2-diamine: 7.81 (d, J=5.8 Hz, 1H), 6.47 (m, 2H), 5.57 (s, br, 1H), 3.08 (m, 2H), 2.77 (m, 2H)

Set b)—N′-(2-methoxy-4-pyridyl)ethane-1,2-diamine: 7.65 (d, J=5.8 Hz, 1H), 6.19 (dd, J=5.9 Hz, 2.1 Hz, 1H), 5.82 (d, J=5.0 Hz, 1H), 5.18 (s br, 1H), 3.77 (s, 3H), 3.08 (m, 2H), 2.77 (m, 2H)

The relative integrals of these sets were 1.0 for set a) and 0.3 for set b).

### Example 2: Synthesis of N′-(2-methoxy-4-pyridyl)ethane-1,2-diamine

No purification of step a) product prior to proceeding to step b), only concentration. Step b) alkoxylation in 2-methyl-THF under pressure.

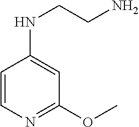

### Step a): N′-(2-chloro-4-pyridyl)ethane-1,2-diamine

2-Chloro-4-nitropyridine (200 g, 1.2 mol) was dissolved in ethanol (2100 ml), ethylenediamine (670 ml, 10 mol) was added over 20 minutes and the mixture was stirred at room temperature overnight. The solution was concentrated under reduced pressure to yield 400 g of a residue that was used directly in the next step.

MS 172.0 (M+1)

### Step b): N′-(2-methoxy-4-pyridyl)ethane-1,2-diamine

The residue obtained in step a) was dissolved in 2-methyl-THF (900 ml), sodium methoxide (202 g, 3.7 mol) was added and the mixture was heated with stirring at 120-130° C. for 20 hours resulting in a pressure of 3-5 bar. After cooling to room temperature water (800 ml) was added, the phases were separated and the aqueous layer was extracted with 2-methyl-THF (3×500 ml). The organic phases were combined, concentrated under reduced pressure, the residue was dissolved in 2-methyl-THF (500 ml). The solution was dried over sodium sulfate and evaporated to dryness to yield 178 g (87% yield for 2 steps).

¹H-NMR (300 MHz, DMSO-d₆): 7.55 (d, J=6.1 Hz, 1H), 6.16 (dd, J=6.1 Hz, 2.0 Hz, 1H), 5.75 (d, J=1.9 Hz, 1H), 3.68 (s, 3H), 3.03 (t, J=6.2 Hz, 2H), 2.65 (t, 3H); MS 168.0 (M+1)

### Example 3: Synthesis of N′-(2-methoxy-4-pyridyl)ethane-1,2-diamine

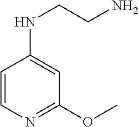

One-pot procedure with anisole as solvent at ambient pressure using sodium methoxide as the reagent for step b).

Anisole (12 ml) was heated to 50° C., ethylenediamine (6 ml, 89 mmol) was added with stirring followed by 2-chloro-4-nitropyridine (2 g, 12.2 mmol). Stirring was continued at 50° C. for two hours. Then sodium methoxide (1.98 g, 36.7 mmol) was added and the mixture was heated to reflux and stirred under reflux overnight.

Water (1 ml) was added to the mixture, which was concentrated under reduced pressure. Water (20 ml) was added to the residue, the mixture was saturated with NaCl and extracted with 2-methyl-THF (3×20 ml). The combined organic layers were washed with brine (2×), dried (MgSO₄), and the solvent was removed under reduced pressure to yield 1.64 g (80% yield).

¹H-NMR (300 MHz, DMSO-d₆): 7.61 (d, J=5.8 Hz, 1H), 6.42 (m, 1H), 6.20 (dd, J=5.9 Hz, 2.0 Hz, 1H), 5.77 (d, J=1.9 Hz, 1H), 3.71 (s, 3H), 2.99 (m, 2H), 2.66 (m, 2H); MS 168.1 (M+1).

The derivatives with 2-ethoxy and 2-n-propoxy were synthesized in the same manner.

| | |
|---|---|
| | Formula (IV) |
| 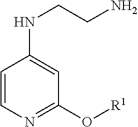 | |

| R₁ | NMR | MS |
|---|---|---|
| OCH₂CH₃ | ¹H-NMR (300 MHz, CD₃CN): 7.63 (d, J = 5.9 Hz, 1H), 6.17 | 182.2 |
| | (dd, J = 5.8 Hz, 2.0 Hz, 1H), 5.78 (d, J = 2.1 Hz, 1H), 5.19 (s | (M + 1) |
| | br, 1H), 4.21 (q, J = 7.1 Hz, 2 H), 3.07 (m, 2H), 2.76 (m, | |
| | 2H), 1.6 (s br, 2H), 1.27 (t, J = 7.1 Hz, 3 H) | |
| OCH₂CH₂CH₃ | 1H-NMR (300 MHz, CD₃CN): 7.63 (d, J = 5.9 Hz, 1H), 6.17 | 196.2 |
| | (dd, J = 5.9 Hz, 2.0 Hz, 1H), 5.79 (d, J = 2.0 Hz, 1H), 5.19 | (M + 1) |
| | (s, 1H), 4.11 (t, J = 6.7 Hz, 2 H), 3.07 (q, J = 5.9 Hz, 2H), | |
| | 2.77 (t, J = 6.2 Hz, 2H), 1.68 (m, 2 H), 1.57 (s, 2H), 0.95 (t, | |
| | J = 7.4 Hz, 3 H) | |

### Example 4: Synthesis of N′-(2-methoxy-4-pyridyl)ethane-1,2-diamine

One-pot procedure with anisole as solvent at ambient pressure using potassium methoxide as the reagent for step b).

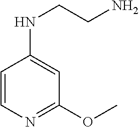

Anisole (12 ml) was heated to 50° C., ethylenediamine (6 ml, 89 mmol) was added with stirring followed by 2-chloro-4-nitropyridine (2 g, 12.2 mmol). Stirring was continued at 50° C. for two hours. Then potassium methoxide (2.57 g, 36.7 mmol) was added and the mixture was heated to reflux and stirred under reflux for 2.5 hours.

Water (1 ml) was added to the mixture, which was concentrated under reduced pressure. Water (20 ml) was added to the residue, the mixture was saturated with NaCl and extracted with 2-methyl-THF (3×20 ml). The combined organic layers were washed with brine (2×), dried (MgSO₄), and the solvent was removed under reduced pressure to yield 1.638 g (80% yield).

¹H-NMR (300 MHz, DMSO-d₆): 7.61 (d, J=5.9 Hz, 1H), 6.42 (m, 1H), 6.20 (dd, J=6.1 Hz, 2.0 Hz, 1H), 5.77 (d, J=1.6 Hz, 1H), 3.71 (s, 3H), 2.99 (m, 2H), 2.66 (m, 2H); MS 168.2 (M+1).

### Example 5: Synthesis of N′-(2-methoxy-4-pyridyl)ethane-1,2-diamine

One-pot procedure with isolation as tosylate salt.

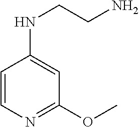

A mixture of ethylenediamine (253 ml, 3.8 mol), potassium carbonate (52.3 g, 0.38 mol) and anisole (300 ml) was heated to 55° C. 2-Chloro-4-nitropyridine (100 g, 0.63 mol) was added in portions over 90 minutes with stirring keeping the temperature at 55° C. Stirring was continued for 75 minutes at the same temperature, then a slurry of potassium methoxide (110.4 g, 1.57 mol) in anisole (300 ml) was added and the resulting mixture was heated at reflux for 2 hours. The temperature was lowered to 55° C., sodium bicarbonate (37.1 g, 0.44 mol) was added in one portion. The temperature was further lowered to room temperature and stirring was continued overnight. The mixture was concentrated under reduced pressure while replacing the distillate (650 ml) with anisole under constant volume. The resulting slurry was filtered, the filter residue was washed with hot anisole (2×200 ml) and the combined filtrates were concentrated under reduced pressure (600 ml distillate). After cooling to room temperature, isopropanol (80 ml) and p-toluenesulfonic acid (91 g, 0.53 mol) were added and the resulting suspension was stirred at room temperature overnight. The mixture was distilled at constant volume replacing the distillate with isopropanol under reduced pressure (200 ml distillate). The suspension was cooled first to room temperature and then to 0° C. The solid was filtered off, washed with isopropanol (140 ml), dried under reduced pressure to yield 161.9 g.

¹H-NMR (600 MHz, CD₃OD): 7.72 (d, J=8.3 Hz, 2H), 7.70 (d, J=6.1 Hz, 1H), 7.25 (d, J=7.7 Hz, 2H), 6.33 (dd, J=6.0 Hz, 2.0 Hz, 1H), 6.00 (d, J=2.0 Hz, 1H), 3.84 (s, 3H), 3.47 (t, J=6.3 Hz, 2H), 3.13 (t, J=6.2 Hz, 2H), 2.39 (s, 3H); MS 168.2 (M+1).

### Example 6: Synthesis of (E)-4,4,4-trifluoro-N-[2-[(2-methoxy-4-pyridyl)amino]ethyl]but-2-enamide

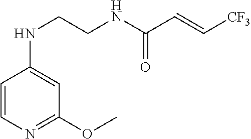 [SMILES: COc1cc(NCCNC(=O)/C=C/C(F)(F)F)ccn1]

### Acylation Via Anhydride.

(E)-4,4,4-Trifluorobut-2-enoic acid (8.4 g, 60 mmol) was dissolved in 2-methyl-THF (150 ml) and cooled to 0° C. TEA (8.4 ml, 60 mmol) was added at 0° C. followed by pivaloyl chloride (6.9 ml, 56 mmol). The resulting mixture was stirred for 90 minutes at room temperature and then cooled to 0° C. N′-(2-Methoxy-4-pyridyl)ethane-1,2-diamine (10 g, 60 mmol) was dissolved in 2-methyl-THF (30 ml) and added dropwise with stirring to the solution of the anhydride at 0° C. After the addition was complete, the ice bath was removed and stirring was continued overnight. The mixture was diluted with water (200 ml), acidified to pH 3.5 with HCl (4M) and washed with 2-methyl-THF (2×20 ml). The aqueous layer was basified to pH 7.5 with NaOH (4M) and extracted with 2-methyl-THF (3×100 ml). The combined extracts were concentrated under reduced pressure to yield 16.2 g (99% yield).

¹H-NMR (300 MHz, CD₃CN): 7.72 (d, J=6 Hz, 1H), 7.1 (s, 1H), 6.7 (m, 1H), 6.23 (dd, J=6 Hz, 2 Hz, 1H), 5.91 (d, J=2 Hz, 1H), 5.2 (s, 1H), 3.81 (s, 3H), 3.44 (m, 2H), 3.28 (m, 2H), MS 290.2 (M+1)

According to this procedure, the following compounds have been synthesized:

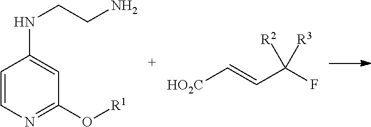

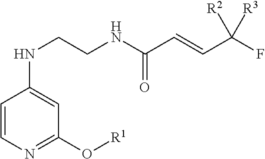

| R¹ | R² | R³ | NMR | MS |
|---|---|---|---|---|
| CH₂CH₃ | F | F | ¹H (300 MHz, CD₃CN) 7.69 (d, J = 6 Hz, 1H), 7.04 | 304.1 |
| | | | (m, 1H), 6.7 (m, 2H), 6.21 (dd, J = 6 Hz, 2 Hz, | |
| | | | 1H), 5.87 (d, 2 Hz, 1H), 5.18 (m, 1H), 4.25 (q, J = | |
| | | | 7 Hz, 2 H), 3.44 (m, 2 H), 3.28 (m, 2H), 1.31 (t, | |
| | | | J = 7 Hz, 3 H) | |
| (CH₂)₂CH₃ | F | F | ¹H (300 MHz, CD₃CN) 7.69 (d, J = 6 Hz, 1H), 7.03 | 318.2 |
| | | | (m, 1H), 6.7 (m, 2H), 6.21 (dd, J = 6 Hz, 2 Hz, | |
| | | | 1H), 5.88 (d, J = 2 Hz, 1H), 5.17 (m, 1H), 4.16 (t, | |
| | | | J = 7 Hz, 2 H), 3.44 (m, 2 H), 3.28 (m, 2H), 1.73 | |
| | | | (m, 2H), 1.00 (t, J = 7 Hz, 3 H) | |
| CH(CH₃)₂ | F | F | ¹H (300 MHz, CD₃CN) 7.68 (d, J = 6 Hz, 1H), 7.09 | 318.2 |
| | | | (s, 1H), 6.7 (m, 2H), 6.19 (dd, J = 6 Hz, 2 Hz), | |
| | | | 5.82 (d, 2Hz, 1H), 5.2 (m, 2H), 3.42 (m, 2 H), | |
| | | | 3.28 (m, 2H), 1.27 (t, J = 6 Hz, 6 H) | |
| CH₂CH₃ | F | CF₃ | ¹H (300 MHz, CD₃CN) 7.68 (d, J = 6 Hz, 1H), 7.24 | 354.1 |
| | | | (m, 1H), 6.7 (m, 2H), 6.24 (d, J = 5 Hz, 1H), 5.90 | |
| | | | (s, 1H), 5.41 (m, 1H), 4.26 (t, J = 7 Hz, 2 H), 3.44 | |
| | | | (m, 2 H), 3.31 (m, 2H), 1.32 (t, J = 7 Hz, 3 H) | |
| CH₂CH₃ | Cl | CF₃ | ¹H (300 MHz, CD₃CN) 7.69 (d, J = 6 Hz, 1H), 7.02 | 370.1 |
| | | | (m, 1H), 6.8 (m, 1H), 6.61 (d, J = 15 Hz, 1H), 6.21 | |
| | | | (dd, J = 6 Hz, 2 Hz, 1H), 5.87 (d, J = 2 Hz, 1H), | |
| | | | 5.17 (m, 1H), 4.25 (q, J = 7 Hz, 2 H), 3.44 (m, 2 | |
| | | | H), 3.28 (m, 2H), 1.31 (t, J = 7 Hz, 3 H) | |
| CH₂CH₃ | OCH₃ | CF₃ | ¹H (300 MHz, CD₃CN) 7.68 (d, J = 6 Hz, 1H), 7.10 | 366.1 |
| | | | (m, 1H), 6.6 (m, 1H), 6.21 (dd, J = 6 Hz, 2 Hz, | |
| | | | 1H), 5.88 (d, J = 2 Hz, 1H), 5.23 (m, 1H), 4.25 (q, | |
| | | | J = 7 Hz, 2 H), 3.50 (d, J = 1 Hz, 3H), 3.46 (m, 2 | |
| | | | H), 3.28 (m, 2H), 1.31 (t, J = 7 Hz, 3 H) | |
| CH₂CH₃ | F | Br | ¹H (300 MHz, CD₃CN) 7.69 (d, J = 6 Hz, 1H), 7.00 | 366.0 |
| | | | (m, 1H), 6.94 (dt, J = 15 Hz, 11 Hz, 1H), 6.44 (dt, | |
| | | | J = 15 Hz, 2 Hz, 1H), 6.21 (dd, J = 6 Hz, 2 Hz, | |
| | | | 1H), 5.87 (d, J = 2 Hz, 1H), 5.17 (m, 1H), 4.25 (q, | |
| | | | J = 7 Hz, 2 H), 3.43 (m, 2 H), 3.28 (m, 2H), 1.31 | |
| | | | (t, J = 7 Hz, 3 H) | |
| CH₂CH₃ | Cl | Cl | ¹H (300 MHz, DMSO-d₆) 8.67 (m, 1H), 7.62 (d, | 336.1 |
| | | | J = 6 Hz, 1H), 7.00 (t, J = 15 Hz, 1H), 6.54 (m, 2 | |
| | | | H), 6.21 (d, J = 5 Hz, 1H), 5.83 (s, 1H), 4.18 (q, | |
| | | | J = 7 Hz, 2 H), 3.30 (m, 2H), 3.17 (m, 2H), 1.24 (t, | |
| | | | J = 7 Hz, 3 H) | |
| CH₂CH₃ | H | CF₂OCH₃ | ¹H (300 MHz, CD₃CN) 7.64 (d, J = 6 Hz, 1H), | 348.1 |
| | | | 6.88 (s, 1H), 6.22 (ddd, J = 5 Hz, 15 Hz, 19 Hz, | |
| | | | 1H), 6.26 (dt, J = 2 Hz, 15 Hz, 1H), 6.17 (dd, J = | |
| | | | 2 Hz, 6 Hz, 1H), 5.83 (d, J = 2 Hz, 1H), 5.27 (m, | |
| | | | 2H), 4.21 (q, J = 7 Hz, 2H), 3.61 (s, 3H), 3.38 (m, | |
| | | | 2H), 3.22 (m, 2H), 1.27 (t, J = 7 Hz, 3 H) | |
| CH₃ | Cl | CF₃ | ¹H (300 MHz, CD₃CN) 7.71 (d, J = 6 Hz, 1H), 7.0 | 356.1 |
| | | | (s, 1H), 6.84 (m, 1H), 6.61 (d, J = 15 Hz, 1H), | |
| | | | 6.23 (dd, J = 2 Hz, 6 Hz, 1H), 5.91 (d, J = 2 Hz, | |
| | | | 1H), 5.2 (m, 1H), 3.81 (s, 3H), 3.44 (m, 2H), 3.28 | |
| | | | (m, 2H) | |
| CH₃ | OCH₃ | CF₃ | ¹H (300 MHz, CD₃CN) 7.71 (d, J = 6 Hz, 1H), 7.0 | 352.1 |
| | | | (s, 1H), 6.59 (m, 1H), 6.23 (dd, J = 2 Hz, 6 Hz, | |
| | | | 1H), 5.91 (d, J = 2 Hz, 1H), 5.2 (m, 1H), 3.81 (s, | |
| | | | 3H), 3.51 (d, J = 1 Hz, 3H), 3.43 (m, 2H), 3.29 | |
| | | | (m, 2H) | |
| CH₃ | Br | F | ¹H (300 MHz, CD₃CN) 7.71 (d, J = 6 Hz, 1H), 7.1 | 352.1 |
| | | | (s, 1H), 6.94 (dt, J = 11 Hz, 15 Hz, 1H), 6.46 (dt, | |
| | | | J = 2 Hz, 15 Hz, 1H), 6.23 (dd, J = 2 Hz, 6 Hz, | |
| | | | 1H), 5.91 (d, J = 2 Hz, 1H), 5.2 (m, 1H), 3.81 (s, | |
| | | | 3H), 3.43 (m, 2H), 3.27 (m, 2H) | |
| CH₃ | F | H | ¹H (300 MHz, CD₃CN) 7.71 (d, J = 6 Hz, 1H), 7.0 | 272.1 |
| | | | (s, 1H), 6.2-6.7 (m, 4H), 5.91 (d, J = 2 Hz, 1H), | |
| | | | 5.2 (m, 1H), 3.81 (s, 3H), 3.43 (m, 2H), 3.27 (m, | |
| | | | 2H) | |
| CH₃ | H | CF₂OCH₃ | ¹H (300 MHz, CD₃CN) 7.71 (d, J = 6 Hz, 1H), 6.9 | 334.1 |
| | | | (s, 1H), 6.66 (ddd, J = 5 Hz, 15 Hz, 19 Hz, 1H), | |
| | | | 6.30 (dt, J = 2 Hz, 15 Hz, 1H), 6.23 (dd, J = 2 Hz, | |
| | | | 6 Hz, 1H), 5.90 (d, J = 2 Hz, 1H), 5.3 (m, 2H), | |
| | | | 3.81 (s, 3H), 3.65 (s, 3H), 3.42 (m, 2H), 3.26 (m, | |
| | | | 2H) | |
| CH₃ | Cl | Cl | ¹H (300 MHz, CD₃CN) 7.65 (d, J = 6 Hz, 1H), | 322.0 |
| | | | 6.66 (dd, J = 14 Hz, 15 Hz, 1H), 6.45 (d, J = 15 | |
| | | | Hz, 1H), 6.25 (dd, J = 2 Hz, 6 Hz, 1H), 5.90 (d, J = | |
| | | | 2 Hz, 1H), 3.78 (s, 3H), 3.44 (s, 3H), 3.42 (m, | |
| | | | 2H), 3.26 (m, 2H) | |

### Example 7: Synthesis of (E)-N-[2-[(2-ethoxy-4-pyridyl)amino]ethyl]-4,4,4-trifluoro-but-2-enamide

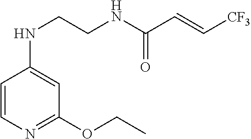 [SMILES: CCOc1cc(NCCNC(=O)/C=C/C(F)(F)F)ccn1]

### Acylation of Salt of Amino Building Block Via Anhydride.

(E)-4,4,4-Trifluorobut-2-enoic acid (1.0 g, 7.14 mmol) was dissolved in 2-methyl-THF (13 ml) under a nitrogen atmosphere and cooled to 0-5° C. Triethylamine (0.995 ml, 7.14 mmol) was added followed by pivaloyl chloride (0.791 ml, 6.43 mmol). The mixture was stirred at ambient temperature for 90 min, then cooled to 0-5° C. and combined with a solution of the napadisylate salt of N′-(2-ethoxy-4-pyridyl)ethane-1,2-diamine (2.32 g, 3.57 mmol) in 2-methyl-THF (16 ml) at 0-5° C. The resulting mixture was stirred at ambient temperature for 2 hours and then diluted with water (25 ml). The mixture was acidified with HCl (10%) to a of pH 3.0, the phases were separated. The aqueous phase was washed with 2-methyl-THF (3×30 ml), alkalized with NaOH (4M) to a pH of 7.5 and extracted with 2-methyl-THF. The combined extracts were concentrated under reduced pressure to yield 1.709 g (87% yield).

¹H-NMR (300 MHz, CD₃CN): 7.69 (d, J=5.9 Hz, 1H), 7.06 (s, 1H), 6.78 (m, 2H), 6.21 (dd, J=5.9 Hz, 2.1 Hz, 1H), 5.87 (d, J=2.0 Hz, 1H), 5.19 (m, 1H), 4.25 (q, J=7.1 Hz, 2H), 3.44 (q, J=6.1 Hz, 2H), 3.28 (q, J=6.1 Hz, 2H), 1.31 (t, J=7.1 Hz, 3H). MS 304.1 (M+1).

### Example 8: Synthesis of (E)-4,4-difluoro-N-[2-[(2-methoxy-4-pyridyl)amino]ethyl]pent-2-enamide

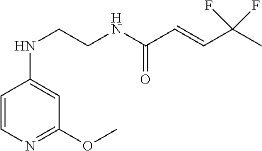 [SMILES: COc1cc(NCCNC(=O)/C=C/C(C)(F)F)ccn1]

Acylation with Propylphosphonic Anhydride (T3P)

(E)-4,4-Difluoropent-2-enoic acid (4.27 g, 31.4 mmol) and N′-(2-methoxy-4-pyridyl)ethane-1,2-diamine (5 g, 29.9 mmol) was dissolved in ethyl acetate (30 ml). Triethylamine (10.4 ml, 74.8 mmol) was added and the mixture was cooled to 0° C. Propylphosphonic anhydride (50% in EtOAc, 18.7 ml, 31.4 mmol) was added over 45 min. The resulting mixture was stirred for 90 minutes. Water (60 ml) was added, and the mixture was acidified with sulfuric acid (4M) to a pH of 3.5 and washed with EtOAc (2×30 ml). The aqueous phase was alkalized to a pH of 7.5 with NaOH (4M) and extracted with EtOAc (3×40 ml). The combined extracts were concentrated under reduced pressure to yield 6.5 g (76% yield).

¹H-NMR (300 MHz, CD₃CN): 7.71 (d, J=5.9 Hz, 1H), 6.89 (s br, 1H), 6.72 (dt, J=11 Hz, 16 Hz, 1H), 6.33 (dt, J=2 Hz, 16 Hz, 1H), 6.23 (dd, J=6 Hz, 2 Hz, 1H), 5.90 (d, J=2 Hz, 1H), 5.21 (s br, 1H), 3.81 (s, 3H), 3.42 (m, 2H), 3.26 (m, 2H), 1.77 (t, J=19 Hz, 3H); MS 286.1 (M+1)

In an analogous way, (E)-4,4,4-trifluoro-N-[2-[(2-methoxy-4-pyridyl)amino]ethyl]but-2-enamide was synthesized starting from (E)-4,4,4-trifluorobut-2-enoic acid with 87% yield.

### Example 9: Synthesis of (E)-4,4-difluoro-N-[2-[(2-methoxy-4-pyridyl)amino]ethyl]pent-2-enamide

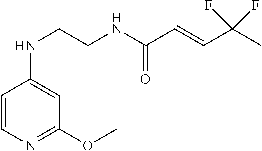 [SMILES: COc1cc(NCCNC(=O)/C=C/C(C)(F)F)ccn1]

Acylation of Tosylate Salt of Amine with T3P

The p-toluenesulfonate salt of N′-(2-methoxy-4-pyridyl)ethane-1,2-diamine (7.5 g, 22.2 mmol), (E)-4,4-difluoropent-2-enoic acid (3 g, 22.2 mmol) and triethylamine (7.86 g, 78 mmol) were charged in ethyl acetate (15 ml) and the resulting slurry cooled in an ice bath. Propane phosphonic acid anhydride (T3P, 50% w/w in ethyl acetate, 14.5 ml, 0.48 mol) was added dropwise over 15 minutes. Stirring was continued for 30 min at 0° C., then the temperature was raised to room temperature and stirring was continued for 45 min. Sulfuric acid (0.4M, 16.7 mmol) was added, the phases were separated, the organic phase was extracted with water (7.5 ml), the aqueous extracts were combined. Activated charcoal (ca 0.15 g) was added, the mixture was concentrated under reduced pressure and filtered. The filtrate was diluted with ethanol and ethyl acetate (6 ml each), the pH was adjusted to 6 with 5M NaOH. The temperature was raised to ca. 40° C., sodium hydroxide (32% w/w, 20 mmol) was added over 5 min. Stirring was continued for one hour at ca. 40° C. and at room temperature, then the suspension was filtered and washed with cold water to yield a solid (5.6 g, 88%).

¹H-NMR (300 MHz, CD₃CN): 7.71 (d, J=6 Hz, 1H), 6.90 (s br, 1H), 6.72 (dt, J=11 Hz, 16 Hz, 1H), 6.33 (dt, J=2 Hz, 16 Hz, 1H), 6.23 (dd, J=6 Hz, 2 Hz, 1H), 5.90 (d, J=2 Hz, 1H), 5.21 (s br, 1H), 3.81 (s, 3H), 3.42 (q, J=6 Hz, 2H), 3.26 (q, J=6 Hz, 2H), 1.77 (t, J=19 Hz, 3H); MS 286.1 (M+1)

1-20. (canceled)

21. A process for producing a compound of Formula (I) 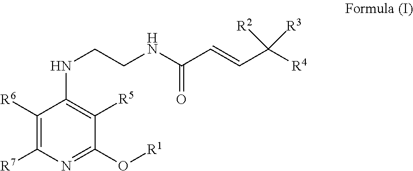
wherein:
R¹ is C₁-C₆ alkyl, wherein the C₁-C₆ alkyl is optionally substituted with halo;
R²-R⁴ are independently H, halo, C₁-C₆ alkyl or C₁-C₆ alkoxy, wherein the C₁-C₆ alkyl and the C₁-C₆ alkoxy are optionally substituted with halo, C₁-C₆ alkyl or C₁-C₆ alkoxy; and
R⁵-R⁷ are independently H or C₁-C₆ alkyl, wherein the C₁-C₆ alkyl is optionally substituted with halo;
comprising
a) reacting a compound of Formula (II) 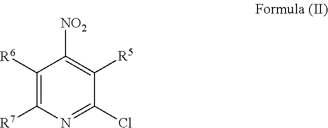
with ethylenediamine to form a compound of Formula (III) 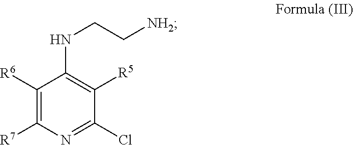
b) reacting in situ the compound of Formula (III) with R¹OX, wherein X is Na or K to form a compound of Formula (IV)

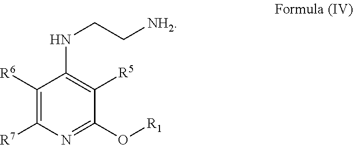

22. The process of claim 21, further comprising
step c) acylating the compound of Formula (IV) to give the compound of Formula (I).

23. The process of claim 21, wherein R⁵, R⁶ and R⁷ are H.

24. The process of claim 21, wherein R¹ is selected from the group consisting of methyl, ethyl, n-propyl and isopropyl.

25. The process of claim 21, wherein R⁴ is F.

26. The process of claim 21, wherein R² and R³ are independently selected from the group consisting of H, halo, CH₃, OCH₃ and CF₂OCH₃.

27. The process of claim 21, wherein R⁵, R⁶ and R⁷ are H, R⁴ is F and R¹, R² and R³ are as defined below:

| R³ | R² | R³ |
|---|---|---|
| CH₃ | F | F |
| CH₂CH₃ | F | F |
| CH₃ | OCH₃ | CF₃ |
| CH₃ | Br | F |
| CH₃ | F | H |
| CH₃ | H | CF₂OCH₃ |
| CH₃ | Cl | Cl |
| CH₃ | F | CH₃ |

28. A process for producing a compound of Formula (I) 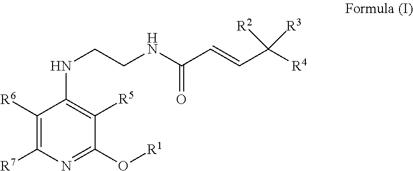
wherein:
R¹ is C₁-C₆ alkyl, wherein the C₁-C₆ alkyl is optionally substituted with halo;
R²-R⁴ are independently H, halo, C₁-C₆ alkyl or C₁-C₆ alkoxy, wherein the C₁-C₆ alkyl and the C₁-C₆ alkoxy are optionally substituted with halo, C₁-C₆ alkyl or C₁-C₆ alkoxy; and
R⁵-R⁷ are independently H or C₁-C₆ alkyl, wherein the C₁-C₆ alkyl is optionally substituted with halo;
comprising
a) reacting a compound of Formula (V) 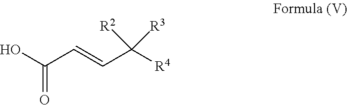
with a compound of Formula (IV) or a salt thereof 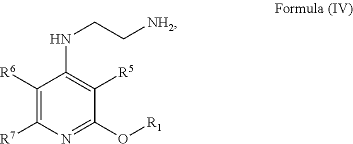
and an activating agent to give the compound of Formula (I).

29. The process of claim 28, wherein the compound of Formula (V) is initially reacted with the activating agent to give a mixed anhydride which is then reacted with the compound of Formula (IV).

30. The process of claim 29, wherein the activating agent is pivaloyl chloride or propylphosphonic anhydride and the mixed anhydride is a compound of formula (VI) or formula (VII), respectively.

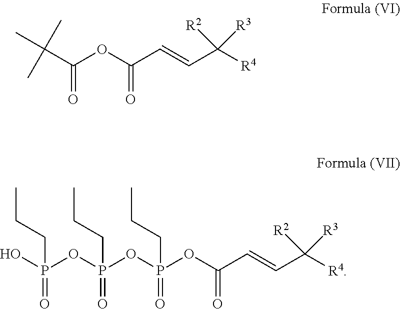

31. The process of claim 28, wherein the compound of Formula (V) and the compound of Formula (IV) are combined before the addition of the activating agent.

32. The process of claim 28, wherein R⁵, R⁶ and R⁷ are H.

33. The process of claim 28, wherein R¹ is selected from the group consisting of methyl, ethyl, n-propyl and isopropyl.

34. The process of claim 28, wherein R⁴ is F.

35. The process of claim 28, wherein R² and R³ are independently selected from the group consisting of H, halo, CH₃, OCH₃ and CF₂OCH₃.

36. The process of claim 28, wherein R⁵, R⁶ and R⁷ are H, R⁴ is F and R¹ is ethyl, and R² and R³ are F.

37. The process of claim 28, wherein R⁵, R⁶ and R⁷ are H, R⁴ is F and R¹ is methyl, and R² is methyl and R³ is F.

38. The process of claim 28, wherein the salt of the compound of Formula (IV) is the hydrochloride, sulfate, oxalate, mesylate, tosylate or napadisylate salt.

39. The process of claim 38, wherein the salt of compound of Formula IV is the napadisylate salt.

40. The process of claim 38, wherein the salt of compound of Formula (IV) is the tosylate salt.
